(19) United States
(12) Patent Application Publication
(10) Pub. No.: US 2008/0312573 A1
(43) Pub. Date: Dec. 18, 2008

(54) Chappell self adhesive medical wrap

(75) Inventor: Mildred L. Chappell, Orlando, FL (US)

Correspondence Address: Mildred L. Chappell, 2122 Tuckaway Court, Orlando, FL, 32826, US

(73) Assignee: Mildred Loween Chappell, Orlando, FL (US)

(21) Appl. No.: 11/811,951

(22) Filed: Jun. 13, 2007

(51) Int. Cl. A61F 13/00; A61L 15/00

(52) U.S. Cl. 602/45; 602/56; 602/77; 602/79

(57) ABSTRACT

A self adhesive medical wrap whose outer layer is moisture impervious. The inside layer is non-woven, moisture pervious and is made of wood cellulose fiber and a superabsorbent material called polyacrylate. Additional synthetic materials include polypropylene, polyester, and polyethylene to reduce leakage from the wound. The edges of the wrap consist of synthetic rubber strands for a closer fit. The bandage consists of three reclosable fasteners. They are the loop and hook system fasteners and contain no adhesives.

### BACKGROUND OF THE INVENTION

The invention pertains to wound care management. More specifically, the invention relates to gaping or seeping wounds or ulcers that require ongoing medical management and wound dressing changes.

The invention possessed benefits and advantages over traditional wound care materials such as tape and gauze as it is more cost effective, easier to use, and uses less materials.

The invention is a self adhesive medical wrap with a moisture impervious outer layer. The inside layer is non-woven, moisture pervious and is comprised of wood cellulose fiber and polyacrylate, a superabsorbant material. The bandage consists of three reclosable loop and hook fasteners.

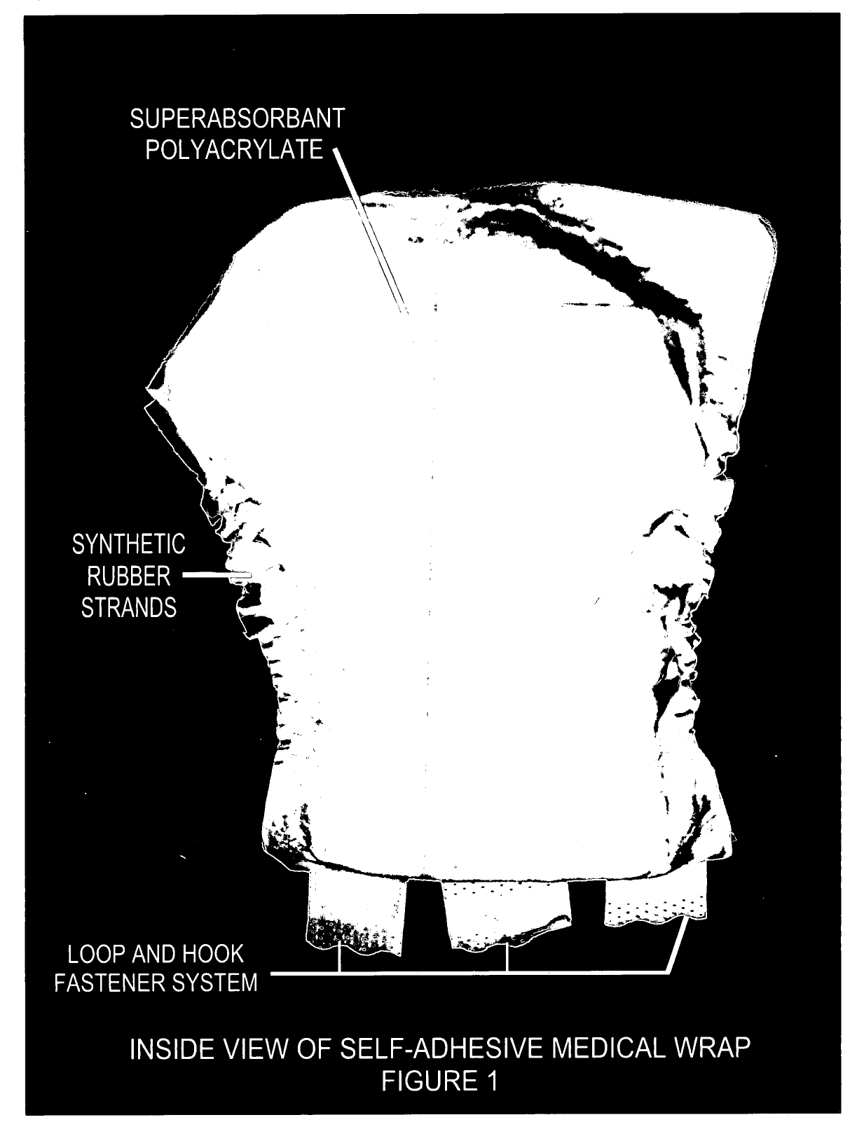

The manner and process of making the bandage material is described as follows: The process starts at the mill, where a sheet of pulp is fed into a rotary mill and is converted into fibers which are transported into a forming pocket using a vacuum generator. As the fibers are produced, they are mixed with a super-absorbent within the drum former. The mix of pulp and powder coming out from the drum is called “the pad” or “the absorbent core”. Once the pad is formed, a layer of tissue (or light weight non-woven) is placed on the top, bottom or around the whole pad. The pad is then compressed using a debulker roll and then it is cut into individual pieces of pad. In the next step, a poly film or cloth-like material is added at the bottom of the pad (or laminated on line) and non-woven material is added at the top. FIG. 1 of the application shows an inside view of the medical wrap calling attention to the superabsorbant polyacrylate core.

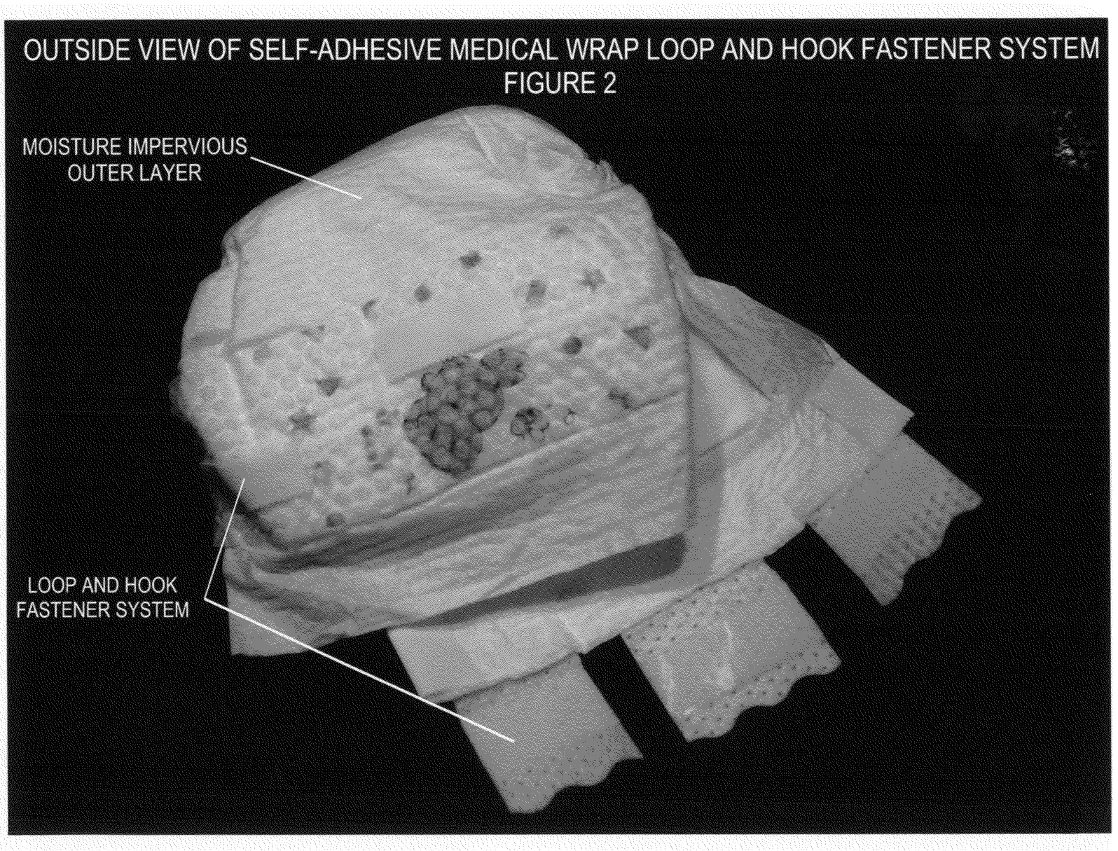

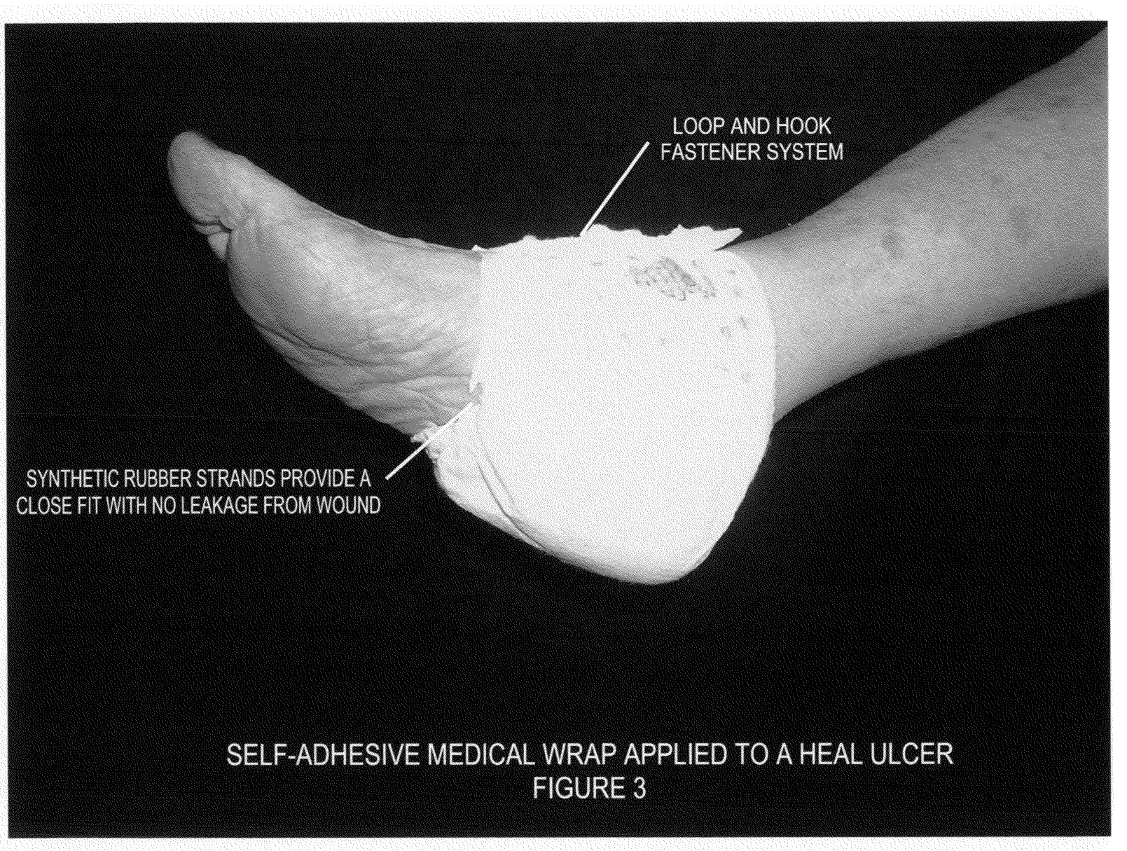

Frontal tape is clued to the poly film or cloth-like backsheet, using a cut and place applicator, before it is added to the bottom of the pad. In order to glue all these materials, hot melt is used in the form of multi-lines or spray. Specialty glue is also used for pad integrity, particularly when the pad is very thin; this help reduce incidence of breaking apart of the bandage when it is wet. Elastomerics are also added at this point to provide stretch to the edges—they are glued with hot melts. Typical elastomerics used are Lycra (Spandex), ppolyurethane or polyesther foam. The top sheet is made of hydrophilic non-woven as shown in FIG. 2 of the application. Also shown in FIGS. 2 and 3 of the application are the synthetic rubber strands, or elastomerics, whose primary function is to provide a close fit without constricting the extremity or allowing leakage.

The next step in the manufacturing process is addition of lateral tapes. They are applied using another cut and place applicator. Tapes can be mechanical tapes, like the hook and loop (originally called and patented as “Velcro”) tapes. After the tapes are added, a die cutting system trims the edge area of the bandage and discards the waste, using a vacuum system. FIGS. 1, 2, and 3 of the application show that there are three separate tabs that comprise the loop and hook fastener system for this medical wrap. The three longer tabs that protrude from the bandage end adhere to the three corresponding receiving tabs located on the outside of the actual bandage. As demonstrated by the food model in FIG. 3, the three tab loop and hook system allows for a close fit of the medical wrap without causing constriction. It also demonstrates the ability of this bandage to be used easily on awkwardly located wounds. Because the loop and hook system is not adhesive and is located on the outer portion of the bandage, there is no concern for skin irritation as the loop and hook system has no physical contact with the skin surface.

1. The wrap absorbs wound seepage without leaking onto clothing garments.

2. There are no adhesives as part of the fastening system which is a loop and hook system; therefore, persons who are allergic to adhesive material will benefit from this type of bandage.

3. Due to the synthetic rubber strands along the outer edges as well as the loop and hook fastening system, the bandage will not slide down or away from the wound.

4. Because the bandage materials are superabsorbent, the wound should heal quicker than with traditional bandaging as the wound will remain dryer which may expedite healing.
